(19) United States
(12) Patent Application Publication
(10) Pub. No.: US 2024/0316595 A1
(43) Pub. Date: Sep. 26, 2024

(54) APPARATUS AND METHOD FOR GENERATING AND TRANSMITTING ULTRASONIC WAVES INTO A TARGET

(71) Applicant: SEDIGN SOLUTIONS PRIVATE LIMITED, Bengaluru (IN)

(72) Inventor: Nikhil Das S, Bangalore (IN)

(21) Appl. No.: 18/575,658

(22) PCT Filed: Jun. 30, 2022
(86) PCT No.: PCT/IN2022/050603

§ 371 Date: Dec. 29, 2023

(30) Foreign Application Priority Data
Jun. 30, 2021 (IN) 202141029490

(51) Int. Cl. B06B 1/06; B08B 7/02

(52) U.S. Cl. CPC B06B 1/0622; B06B 2201/76; B08B 7/028

(57) ABSTRACT

An embodiment herein provides an apparatus for generating and transmitting ultrasonic waves into a non-planar target for one or more applications. The apparatus includes a control system that generates one or more electrical signals, and a transducer assembly including one or more transducers that receive and convert the one or more electrical signals into the ultrasonic waves. The generated ultrasonic waves are efficiently transmitted into the non-planar target in one or more directions by reducing surface mismatch, where the ultrasonic waves cause at least one of an acoustic streaming, a cavitation, a microstreaming, standing waves, a turbulence in a flow of fluids, a vibration of fluid molecules, a vibration of solids, reflection, refraction, or absorption, thereby improving efficiency of the one or more applications comprising any of, but not limited to, cleaning, imaging, mixing, measuring, sensing, or therapy.

### BACKGROUND

### Technical Field

The embodiment herein generally relates to ultrasonic waves, more particularly relates to an apparatus and method for generating and transmitting ultrasonic waves into a target to improve efficiency of one or more applications employing ultrasonic waves.

### Description of Related Art

Sound waves of frequencies higher than 20,000 hertz are called as ultrasonic waves. The ultrasonic waves travelling through a fluid comprises of positive pressure zones and negative pressure zones. Cavities are formed within the fluid due to these pressure variations. Continued exposure of the ultrasonic waves to these cavities leads to their growth in size and sometimes collapse causing shockwaves. This process is known as cavitation and is usually seen when the ultrasonic waves are of low frequencies. When the ultrasonic waves are of higher frequencies, the cavities oscillate but do not collapse. The creation of swift currents in the fluid in the vicinity of these oscillating cavities is known as microstreaming. Transfer of energy from the ultrasonic waves to the fluid particles also causes bulk motion of the fluid known as acoustic streaming. A standing wave is one that is formed by the combination of two waves moving in opposite directions, but having equal frequency and amplitude. A standing wave may be formed when a transmitted wave and a reflected wave interfere within a given, finite space.

A ‘target’ is defined as an object, living or non-living, into which the ultrasonic waves need to be transmitted. It is desirable to transmit ultrasonic waves into a target for applications such as cleaning, imaging, mixing, measuring, sensing, therapy, etc. These applications depend on the waves causing at least one of (i) an acoustic streaming, (ii) a cavitation, (iii) a microstreaming, (iv) standing waves, (v) a turbulence in the flow of fluids, (vi) a vibration of the fluid molecules, (vii) a vibration of solids, (viii) reflection, (ix) refraction, or (x) absorption.

A ‘planar surface’ is defined as a surface on which if any two points are chosen, a straight line joining them lies wholly on that surface. A flat plate, for example, is planar in nature whereas a curved surface of a cylinder or a cone is non-planar. A surface of the target which is available for introducing the ultrasonic waves into the target is defined as a ‘target surface’. The targets with non-planar target surfaces are known as non-planar targets. For example, the curved surfaces of cylinders and cones are non-planar target surfaces; hence, the cylinders and cones are non-planar targets.

Several processes require transmission of the ultrasonic waves into non-planar targets. An example of such a process is an ultrasound imaging which requires the ultrasonic waves to be transmitted into a human body in order to produce images of structures present inside the human body. An ultrasound probe is place on an external surface of the human body which is the target surface in this case, which is non-planar in nature. Non-destructive examination (NDE), another example, uses short, high frequency ultrasonic waves to identify flaws in a target. The target surface in this case may be non-planar if the target is, say, a cylindrical pipe. Ultrasonic cleaning uses ultrasonic waves passing through water to create cavitation that removes contamination from surfaces. Stirring and mixing of liquids may also be achieved by using the ultrasonic waves. The target surface for ultrasonic cleaning and mixing may be non-planar if they are performed in containers which are in the shape of, but not limited to a hemisphere, a cylinder, a frustum, or a cone.

During hemodialysis, a filter unit with a semi-permeable membrane is used to purify the blood from a kidney patient's body. Accumulation of particles like toxins, blood proteins etc. on the membrane leads to reduction in the efficiency of hemodialysis. This reduction leads to incomplete removal of toxins and fluid from the patient's body, leading to a condition called as dialysis inadequacy which puts the patient at risk of developing severe medical complications. The ultrasonic waves may be transmitted into the filter unit to reduce the accumulation of particles and to promote better diffusion of toxins in order to prevent dialysis inadequacy. The process may be termed as an ‘ultrasonic hemodialysis’.

During peritoneal dialysis, a peritoneal membrane in the patient's body acts as a semi-permeable membrane to purify the blood by removing the toxins and excess water. Accumulation of particles on the surface of the peritoneal membrane leads to reduction in the efficiency of peritoneal dialysis. The ultrasonic waves may be transmitted into the patient's abdomen during the peritoneal dialysis, to reduce the accumulation of particles on the peritoneal membrane and to promote better diffusion of toxins in order to prevent the dialysis inadequacy. The process may be termed as an ‘ultrasonic peritoneal dialysis’. The ultrasonic hemodialysis and the ultrasonic peritoneal dialysis may be collectively termed as an ‘ultrasonic dialysis’.

Exchange of particles through membranes may happen at a smaller scale. In an implantable or a wearable artificial kidney, for example, a device is attached to a patient's body which filters the blood continuously or intermittently. The necessity of making it implantable or wearable requires it to be of small size. In some embodiments, the artificial kidney uses nanopore membranes which are fabricated using a nano-fabrication and a microfabrication process. Silicon nanopore membranes (SNM) are used in some embodiments. The ultrasonic waves may be transmitted into artificial kidneys to promote the diffusion and prevent the accumulation of particles on these membranes.

An ‘active element’ is a component or a combination of components which converts electrical signals into the ultrasonic waves. A ‘control system’ generates the electrical signals which are sent to the active element. The active element may use these electrical signals to generate the ultrasonic waves using but not limited to, a piezoelectric effect, a magnetostriction, a Lorentz force, etc. The active element may include, but not limited to, materials like ceramics, polymers, crystals, composites, metals, or a combination thereof. The active element includes, but not limited to, at least one of a piezoelectric crystal, a piezoelectric ceramic, a piezoelectric polymer, a Lead Zirconium Titanate (PZT), a polyvinylidene difluoride (PVDF), a capacitive micromachined ultrasonic transducers (cMUT), or a piezoelectric micromachined ultrasonic transducers (pMUT).

The active element may be bonded to another material defined as a ‘base’. The combination of the active element and the base forms an ‘ultrasonic transducer’. When the active element is supplied with the electrical signals, it generates the ultrasonic waves which are transmitted through the base. The base is selected such that it provides minimal resistance to the propagation of ultrasonic waves.

A ‘holder’ is a structure that comprises of one or more ultrasonic transducers that enables positioning of each of the one or more ultrasonic transducers around the target.

A combination of the one or more ultrasonic transducers with their respective holders, is called as a transducer assembly.

FIG. 1A illustrates a perspective view of a transducer 100 formed by a combination of an active element 102 and a base 104. FIG. 1B illustrates a perspective view of the transducer 100 of FIG. 1A which is turned by 180 degrees. The transducer 100 is an ultrasonic transducer. When a control system (not shown), sends electrical signals to the active element 102, it generates ultrasonic waves which pass through the base 104 and emit out from the other side shown by a hatched area in FIG. 1B. The hatched area which transmits ultrasonic waves may be defined as a transmitting surface 106. Its area is comparable to that of the area of the active element 102 when the base 104 is a planar surface and has low thickness. The active element 102 is behind the transmitting surface 106. The transmitting surface 106, thus, becomes a source of ultrasonic waves when the active element 102 is supplied with electrical signals by the control system.

FIG. 2A illustrates an exploded view of the system 200 with a transducer assembly 202 and a target 204. The ultrasonic waves are generated and transmitted by the transducer assembly 202 into the target 204 when the transducer assembly 202 and the target 204 are combined together as shown by FIG. 2B. It illustrates a front view of the system 200 of FIG. 2A. The target 204 may be a filter unit. An active element 206 bonded to a base 208 forms a transducer 218. The transducer assembly 202 may include one or more ultrasonic transducers 218 and a holder (not shown) for transmitting the ultrasonic waves into the target 204. The target 204 is a cylindrical object with a target surface 210. A section of the base 208 of width equal to ‘ab’ acts as a transmitting surface 212 from which the ultrasonic waves emit out. The area of the transmitting surface 212 is comparable to the area of the active element 206 when the thickness of the base 208 is low.

In applications where the area of the target surface 210 is significantly larger than the area of the transmitting surface 212, it is not sufficient to use a single active element 206. In case of the ultrasonic peritoneal dialysis, use of a single active element 206 with the transmitting surface 212 on the patient's abdomen transmits the ultrasonic waves to only a small section of the peritoneal membrane. Such a low input of ultrasonic energy into the body is not sufficient to effectively increase the adequacy of dialysis. There is a need for covering a larger area of the target 204 for the exposure of ultrasonic waves.

In case of the ultrasonic hemodialysis, the filter unit consists of a dense bundle of hollow-fiber membranes. The target surface 210 of the filter unit is curved, and hence, non-planar. The ultrasonic waves sent from one transducer do not reach all the sections of the filter unit. There is a need for transmitting the ultrasonic waves from more than one direction into the filter unit. When the one or more ultrasonic transducers are used to transmit the ultrasonic waves into the target 204 (i.e. a non-planar target) from more than one direction, there remains a need for a holder to accommodate these one or more ultrasonic transducers. When the holder is constructed in such a way that it covers the target surface 210 partially or fully, it is said to ‘envelop’ the target 204. There is a need for a holder that envelops the target 204 to position the one or more ultrasonic transducers around the target 204.

In processes which run for longer durations, such as the ultrasonic dialysis, if the position of the transducer assembly 202 is not changed, the same sections of the target 204 will receive the ultrasonic waves throughout the process. It is desirable to intermittently change the position of the transducer assembly 202 in order to expose different sections of the target 204 to the ultrasonic waves. It also ensures that more area of the target 204 gets the ultrasonic energy exposure.

The ultrasonic waves travel poorly through air. When a space 216 between the transmitting surface 212 and the target surface 210 (hatched in FIG. 2B) is modified to achieve faithful transmission of ultrasonic waves, the transducer assembly 202 is said to be ‘coupled’ with the target 204. A ‘coupling medium’ (not shown) is usually used to fill the space 216 between the transmitting surface 212 and the target surface 210 to ensure faithful transmission of the ultrasonic waves by elimination of air between the surfaces. An ‘incident surface’ 214 (hatched in FIG. 2A and shown by a section ‘ef’ in FIG. 2B) of the target surface 210 is defined as a surface through which the ultrasonic waves enter the target 204, when the transducer assembly 202 is coupled with the target 204. A ‘surface mismatch’ is defined as a condition wherein a surface area of the incident surface 214 is not equal to that of the transmitting surface 212. A surface mismatch results from improper coupling which may occur due to various reasons.

The surface mismatch may result from a difference in geometry of the transmitting surface 212 and the incident surface 214. A larger space 216 may result when there is a surface mismatch. For example, when the transmitting surface 212 (a planar surface) is coupled with the target surface 210 (non-planar surface), the resultant surface area of the incident surface 214 is larger than that of the transmitting surface 212, resulting in the surface mismatch. When there is a larger space 216 between the transmitting surface 212 and the incident surface 214, due to the surface mismatch, more coupling medium is required to fill the larger space 216. The more the ultrasonic waves travel through the coupling medium, the more is the attenuation of ultrasonic energy. It is therefore desired that surface mismatch due to the difference in the geometry of the transmitting surface 212 and the incident surface 214 is reduced for efficient transmission of the ultrasonic waves into the target 204.

The surface mismatch may arise due to positioning error. The transducer assembly 202 may be positioned in such a way that the transmitting surface 212 is not aligned well with the target surface 210. In this case, upon coupling, the surface area of the resulting incident surface 214 is not equal to that of the transmitting surface 212. As a result, a significant portion of the ultrasonic waves emitted from the transmitting surface 212 do not enter the target 204. There is a need for transducer assembly designs which minimize positioning errors to enable efficient transmission of the ultrasonic waves into the target 204.

If the transmitting surface 212 is not sufficiently pressed against the target surface 210, a larger space 216 may develop between them, leading to improper coupling between the target surface 210 and the transmitting surface 212. An improper coupling results in lesser transmission of the ultrasonic waves into the target surface 210 which implies a reduced surface area of the incident surface 214 compared to that of the transmitting surface 212. Thus, insufficiently pressed transmitting surface 212 against the target surface 210, leads to the surface mismatch. In applications of ultrasound imaging and conventional NDE, technicians push the transducer assembly 202 against the target surface 210 to reduce the surface mismatch. If a similar effect is desired in the absence of a technician, it is necessary to ensure that the transducer assembly 202 (including one or more ultrasonic transducers) is assembled around the target 204 such that the transmitting surface 212 is sufficiently pressed against the target surface 210.

There may be processes which require a prolonged, continuous or intermittent, exposure of ultrasound to the target 204. An implant in the human body requiring exposure to the ultrasonic waves, for example, is expected to run for a long time without the need for any human intervention. The ultrasonic dialysis session lasts for a few hours wherein a technician or patient cannot be expected to keep the one or more transducers in place throughout the process. Long duration transmission of the ultrasonic waves into the target 204 requires ways of fastening the transducer 218 to the target 204, so as to minimize the need for human intervention.

In case of applications requiring transmission of the ultrasonic waves from multiple directions into the target 204, it is inconvenient and cumbersome for the technician to keep the multiple ultrasonic transducers (i.e. the transducer assembly 202) in place around the target 204. There is a need for ways of fastening the transducer 218 to the target 204 to enable entry of the ultrasonic waves from multiple directions.

Hence, there is a need for an apparatus and method for generating and efficiently transmitting ultrasonic waves into a target to improve the efficiency of one or more applications employing ultrasonic waves.

### SUMMARY

An embodiment herein provides an apparatus for generating and transmitting ultrasonic waves into a non-planar target for one or more applications. The apparatus includes a control system and a transducer assembly. The control system generates one or more electrical signals. The transducer assembly includes one or more transducers that receive the one or more electrical signals from the control system and convert the one or more electrical signals into the ultrasonic waves. By reducing surface mismatch, the generated ultrasonic waves are efficiently transmitted into the non-planar target in one or more directions, where the ultrasonic waves cause at least one of (i) an acoustic streaming, (ii) a cavitation, (iii) a microstreaming, (iv) standing waves, (v) a turbulence in a flow of fluids, (vi) a vibration of fluid molecules, (vii) a vibration of solids, (viii) reflection, (ix) refraction, or (x) absorption, thereby improving efficiency of the one or more applications including any of, but not limited to, cleaning, imaging, mixing, measuring, sensing, or therapy.

In some embodiments, the apparatus includes a holder that envelops the non-planar target and positions each of the one or more transducers around the non-planar target to enable entry of the ultrasonic waves from one or more directions into the non-planar target.

In some embodiments, a surface geometry of the transducer assembly is matched with a surface geometry of a target surface in order to achieve a surface match ratio close to 1.

In some embodiments, the one or more transducers includes one or more active elements. A size of the one or more active elements is limited in order to achieve a surface match ratio close to 1.

In some embodiments, the one or more active elements are positioned on the holder that enables transmitting surfaces to contact with a target surface when the transducer assembly is combined with the non-planar target.

In some embodiments, when the transducer assembly is combined with the non-planar target, the transmitting surfaces press against the target surface in order to achieve a surface match ratio close to 1.

In some embodiments, the holder functions as a base with one or more active elements bonded to the holder.

In some embodiments, the holder fastens the one or more transducers to the non-planar target.

In some embodiments, a target surface functions as a base with one or more active elements bonded to the target surface.

In another aspect, an embodiment herein provides a method for generating and transmitting ultrasonic waves into a non-planar target for one or more applications. The method includes generating one or more electrical signals using a control system. The method includes receiving and converting the one or more electrical signals into the ultrasonic waves using a transducer assembly, where the generated ultrasonic waves are efficiently transmitted into the non-planar target in one or more directions by reducing surface mismatch, that causes at least one of (i) an acoustic streaming, (ii) a cavitation, (iii) a microstreaming, (iv) standing waves, (v) a turbulence in a flow of fluids, (vi) a vibration of fluid molecules, (vii) a vibration of solids, (viii) reflection, (ix) refraction, or (x) absorption, thereby improving efficiency of the one or more applications comprising any of, but not limited to, cleaning, imaging, mixing, measuring, sensing, or therapy.

These and other aspects of the embodiments herein will be better appreciated and understood when considered in conjunction with the following description and the accompanying drawings. It should be understood, however, that the following descriptions, while indicating preferred embodiments and numerous specific details thereof, are given by way of illustration and not of limitation. Many changes and modifications may be made within the scope of the embodiments herein without departing from the spirit thereof, and the embodiments herein include all such modifications.

### BRIEF DESCRIPTION OF THE DRAWINGS

The embodiments herein will be better understood from the following detailed description with reference to the drawings, in which:

FIG. 1A illustrates a perspective view of a transducer formed by a combination of an active element and a base according to the prior art;

FIG. 1B illustrates a perspective view of the transducer of FIG. 1A which is turned by 180 degrees according to the prior art;

FIG. 2A illustrates an exploded view of a system with a transducer assembly and a target according to the prior art;

FIG. 2B illustrates a front view of the system of FIG. 2A with the transducer assembly and the target combined together according to the prior art;

FIGS. 3A and 3B illustrate a front view and a side view of an apparatus for generating and transmitting ultrasonic waves for one or more applications according to some embodiments herein;

FIGS. 4A and 4B illustrate exemplary top view and perspective view of the apparatus including a transducer assembly combined with a non-planar target according to some embodiments herein;

FIG. 5A illustrates an exemplary view of an embodiment of the apparatus of FIG. 3A according to some embodiments herein;

FIG. 5B illustrates an exemplary perspective view of the apparatus of FIG. 5A including a transducer assembly that is separated from a non-planar target according to some embodiments herein;

FIG. 6 illustrates an exemplary top view of the apparatus including a transducer assembly combined with a non-planar target according to some embodiments herein;

FIG. 7A illustrates an exemplary top view of an embodiment of a transducer assembly according to some embodiments herein;

FIG. 7B illustrates a perspective view of the transducer assembly of FIG. 7A according to some embodiments herein;

FIGS. 8A and 8B illustrate exemplary side views of a non-planar target surface combined with a transducer assembly according to some embodiments herein; and

FIG. 9 illustrates a method of generating and transmitting ultrasonic waves into a non-planar target for one or more applications according to some embodiments herein.

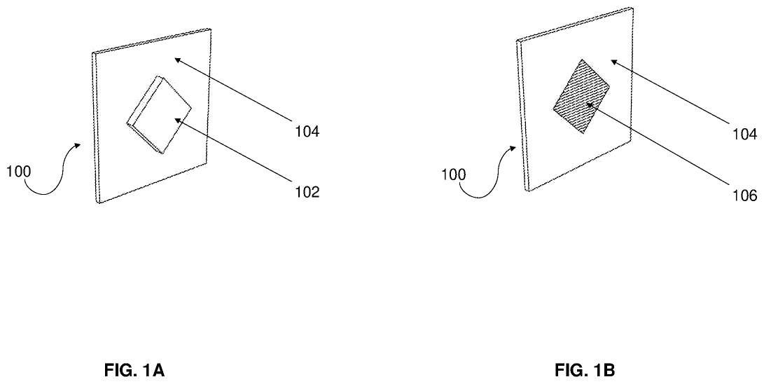

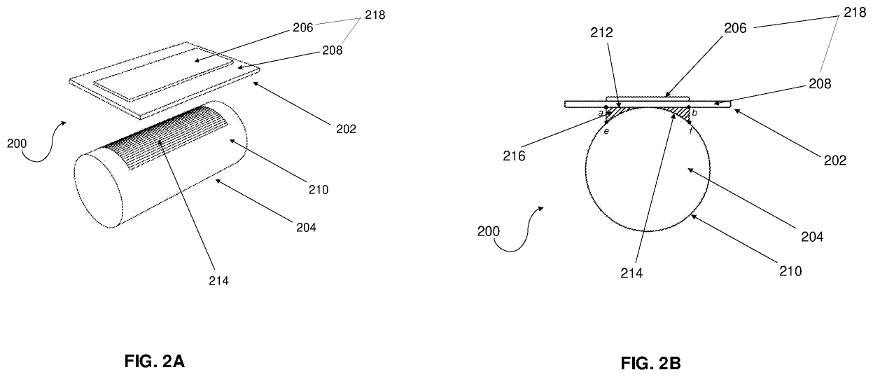

### DETAILED DESCRIPTION OF PREFERRED EMBODIMENTS

The embodiments herein and the various features and advantageous details thereof are explained more fully with reference to the non-limiting embodiments that are illustrated in the accompanying drawings and detailed in the following description. Descriptions of well-known components and processing techniques are omitted so as to not unnecessarily obscure the embodiments herein. The examples used herein are intended merely to facilitate an understanding of ways in which the embodiments herein may be practiced and to further enable those of skill in the art to practice the embodiments herein. Accordingly, the examples should not be construed as limiting the scope of the embodiments herein.

As mentioned, there remains a need for an apparatus and method for generating and efficiently transmitting ultrasonic waves into a target to improve the efficiency of one or more applications employing ultrasonic waves.

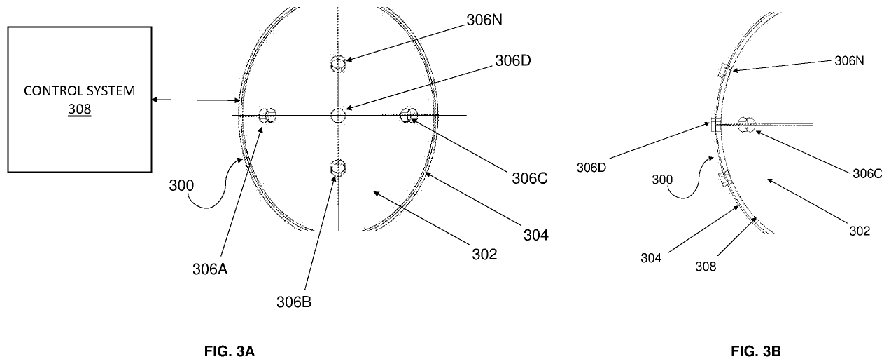

FIGS. 3A and 3B illustrate a front view and a side view, respectively, of an apparatus for generating and transmitting ultrasonic waves for one or more applications according to some embodiments herein. The apparatus includes a control system 308, and a transducer assembly 300. The apparatus generates and transmits the ultrasonic waves into a non-planar target 302 for the one or more applications. The control system 308 is configured to generate one or more electrical signals. The transducer assembly 300 includes one or more transducers 306A-N that receive the one or more electrical signals from the control system 308 and convert the one or more electrical signals into the ultrasonic waves. By reducing surface mismatch, the generated ultrasonic waves are efficiently transmitted into the non-planar target 302 in one or more directions. The ultrasonic waves cause at least one of (i) an acoustic streaming, (ii) a cavitation, (iii) a microstreaming, (iv) standing waves, (v) a turbulence in a flow of fluids, (vi) a vibration of fluid molecules, (vii) a vibration of solids, (viii) reflection, (ix) refraction, or (x) absorption, thereby improving efficiency of the one or more applications. In some embodiments, the one or more applications include, but not limited to, cleaning, imaging, mixing, measuring, sensing or therapy. In an application of an ultrasonic peritoneal dialysis, the transducer assembly 300 transmits ultrasonic waves into the non-planar target 302. The non-planar target 302 may be a patient's abdomen. The ultrasonic waves increase a rate of removal of toxins from the patient's blood, reduce the accumulation of particles on a peritoneal membrane of the patient and increase a turbulence in a dialysis fluid in the peritoneal cavity to promote diffusion of toxins. The word ‘turbulence’ used in this disclosure means ‘randomness’ or ‘disorder’ in the flow of fluids. It is not intended to quantify the degree of randomness in the fluid unlike in the fields of specialization like hydraulics or fluid dynamics.

The non-planar target 302 (i.e. the patient's abdomen) includes a large surface area where the transducer assembly 300 including the one or more transducers 306A-N are positioned around the non-planar target 302. The one or more transducers 306A-N may be one or more ultrasonic transducers. The one or more transducers 306A-N increase an exposure of the ultrasonic waves to the peritoneal membrane. The apparatus may include a holder 304 that envelops the non-planar target 302 and positions each of the one or more transducers 306A-N around the non-planar target 302 to enable entry of the ultrasonic waves into the non-planar target 302 from one or more directions. The holder 304 may be fabricated with materials like, but not limited to, a fabric, a rubber, a metal or a combination thereof to enable it to envelop the non-planar target 302. In some embodiments, the holder 304 comprises any of, but not limited to, hooks, adhesive, belts, Velcro, and the like, to fasten the one or more transducers 306A-N to the non-planar target 302.

The one or more transducers 306A-N transmitting the ultrasonic waves into a target surface 308 from the one or more directions may create a mild turbulence within the non-planar target 302 i.e. the patient's abdomen, causing better removal of toxins from the blood of the patient, hence preventing dialysis inadequacy. In some embodiments, the holder 304 can be moved intermittently with respect to the target surface 308 to change the position of the one or more transducers 306A-N. The intermittent change of the position of the transducer assembly 300 exposes different parts of the peritoneal membrane to the ultrasonic waves and promotes diffusion of toxins. The holder 304 may be moved manually or electrically. In some embodiments, the control system 308 is configured to move the holder 304 to change the position of the one or more transducers 306A-N. The control system 308 may move the holder 304 for every pre-determined time to expose the ultrasonic waves to all the parts of the non-planar target 302.

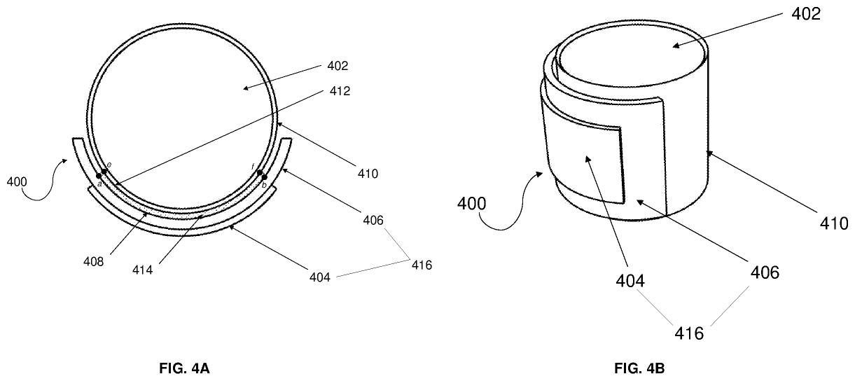

FIGS. 4A and 4B illustrate exemplary top view and perspective view of the apparatus including a transducer assembly 400 combined with a non-planar target 402 according to some embodiments herein. The non-planar target 402 may be a cylindrical container used for ultrasonic mixing of liquids. An active element 404 is bonded to a base 406 to form a transducer 416. The transducer assembly 400 comprises of one or more transducers 416 and a holder (not shown). In some embodiments, the active element 404 is a piezoelectric crystal and the base 406 is a metallic component. The transducer assembly 400 may be shaped in a way that there is a minimal space between a transmitting surface 408 (i.e. section “ab”) and a target surface 410. In some embodiments, the shaping is achieved by matching a surface geometry of the transducer assembly 400 (i.e. including the active element 404 and the base 406) and the target surface 410. In some embodiments, the radii of curvature of the active element 404, the base 406 and the target surface 410 are matched, such that a surface area of the transmitting surface 408 is nearly equal to that of the incident surface 412 (i.e. section “ef”), reducing surface mismatch. The minimal space requires less volume of a coupling medium 414 to be used between the transmitting surface 408 and the incident surface 412 thereby, increasing the efficiency of transmission of the ultrasonic waves.

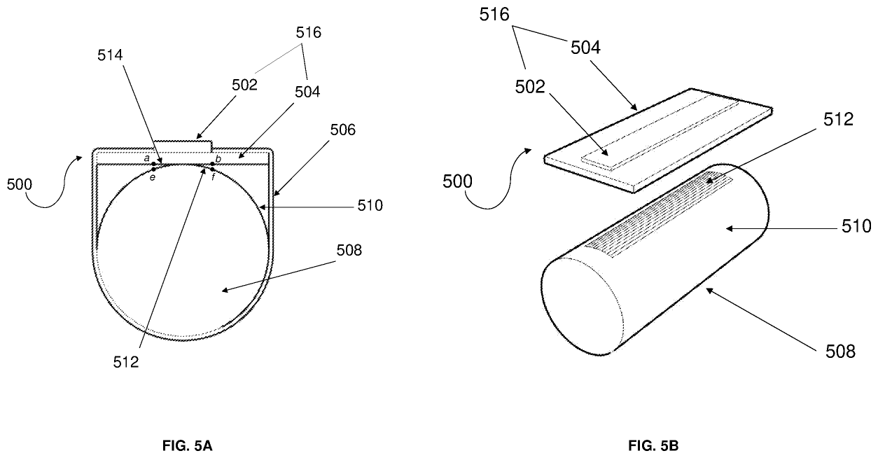

FIG. 5A illustrates an exemplary view of an embodiment of the apparatus of FIG. 3A according to some embodiments herein. The apparatus includes a transducer assembly 500 combined with a non-planar target 508. An active element 502 is bonded to a base 504 to form a transducer 516. A holder 506 enables positioning of the transducer 516 around the non-planar target 508. The combination of the transducer 516 and the holder 506 forms the transducer assembly 500. In some embodiments, the holder 506 fastens the transducer 516 to the non-planar target 508 using mechanisms of fastening like but not limited to, belts, a Velcro, or elastic bands. Upon coupling of the non-planar target 508 with the transducer assembly 500, a ratio of a surface area of a resultant incident surface 512 (i.e. section “ef”) to a surface area of a transmitting surface 514 (i.e. section “ab”) may be defined as a ‘surface match ratio’. In some cases, it is not feasible to fabricate the transducer assembly 500 to match a surface profile of a target surface 510. In such cases, a size of the active element 502 is limited in such a way that when the transducer assembly 500 is coupled with the non-planar target 508, the surface match ratio is between 0.5 and 1.5. The closer the surface match ratio to 1, the lower is the surface mismatch, resulting in more efficient transmission of the ultrasonic waves into the non-planar target 508.

FIG. 5B illustrates an exemplary perspective view of the apparatus of FIG. 5A including the transducer 516 that is separated from the non-planar target 508 according to some embodiments herein. The perspective view depicts the incident surface 512 clearly. The surface area of the transmitting surface 514 (not shown) is approximately same as that of the active element 502. In some embodiments, the active element 502 is a rectangular piezoelectric crystal whose breadth is 35 mm and length is 50 mm. The non-planar target 508 may be with 40 mm diameter and 70 mm length. In such a case, the surface area of the transmitting surface 514 is 1750 sq.mm approximately. The surface area of the incident surface 512, resulting from coupling, is 2125 sq.mm approximately. The surface match ratio is 1.21 (resulting from dividing 2125 by 1750). When the size of the active element 502 is limited to a breadth of 15 mm and a length of 50 mm, the surface area of the transmitting surface is 750 sq.mm approximately. The surface area of the incident surface 512, resulting from coupling, is 768.8 sq.mm approximately. The surface match ratio in this case is 1.02 (resulting from dividing 768.8 by 750). As a result of limiting of the size of the active element by reducing its breadth, a surface match ratio closer to 1 is achieved. It reduces the surface mismatch and ensures more efficient transmission of ultrasonic waves into the non-planar target 508. In some embodiments, the non-planar target 508 is a spherical object with a diameter of 40 mm and the active element 502 is a cylindrical disc of diameter 20 mm. The surface area of the transmitting surface 514 (not shown) is 314.1 sq.mm approximately and the surface area of the incident surface 512 is 336.7 sq.mm approximately. The surface match ratio is 1.07, within the range of 0.5 and 1.5 which implies low surface mismatch. The surface match ratio of the embodiment mentioned in the FIGS. 4A and 4B is very close to 1 as the surface geometry of the transducer assembly 400 is matched with the surface geometry of the target surface 410.

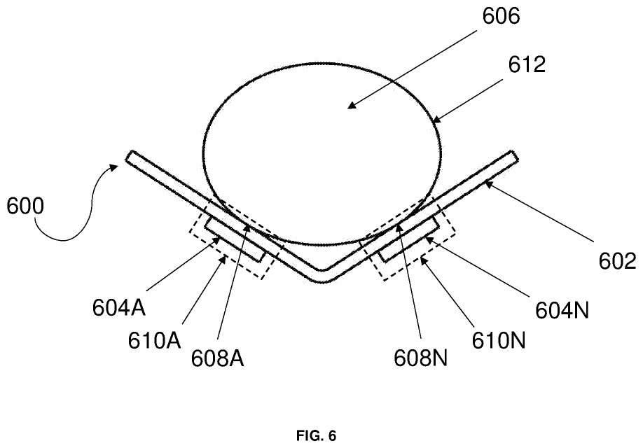

FIG. 6 illustrates an exemplary top view of the apparatus including a transducer assembly 600 combined with a non-planar target 606 according to some embodiments herein. The transducer assembly 600 includes a holder 602 which envelops the non-planar target 606. The holder 602 may function as a base as the one or more active elements 604A-N are bonded to it, forming one or more transducers 610A-N, as shown within the dotted boxes in the FIG. 6. The one or more transducers 610A-N and the holder 602, together, are termed as the transducer assembly 600. The holder 602 enables positioning of each of the one or more transducers 610A-N around the non-planar target 606. The one or more active elements 604A-N are positioned on the holder 602 such that their respective transmitting surfaces 608A-N contact a target surface 612 when the transducer assembly 600 is combined with the non-planar target 606. In some embodiments, the non-planar target 606 is an elliptic cylinder and the holder 602 includes flat surfaces. When a curved surface of a cylinder touches a flat surface, the two surfaces make contact along a line. The one or more active elements 604A-N are positioned on the holder 602 along these lines of contact to ensure that their respective transmitting surfaces 608A-N contact the target surface 612 when the transducer assembly 600 is combined with the non-planar target 606, thereby reducing surface mismatch by minimizing positioning errors to enable efficient transmission of the ultrasonic waves into the non-planar target 606. The size of the one or more active elements 604A-N is limited in order to achieve a surface match ratio further close to 1.

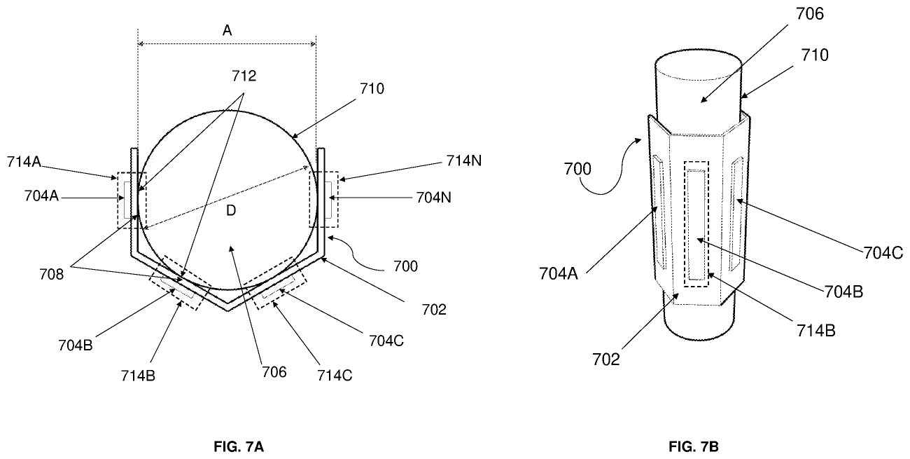

FIG. 7A illustrates an exemplary top view of an embodiment of a transducer assembly 700 according to some embodiments herein. The transducer assembly 700 combines with a non-planar target 706. A holder 702 functions as a base on which one or more active elements 704A-N are bonded, forming one or more transducers 714A-N, as shown within the dotted boxes in the FIG. 7A. The one or more transducers 714A-N and the holder 702, together, are termed as the transducer assembly 700. The holder 702 enables positioning of each of the one or more transducers 714A-N around the non-planar target 706, enveloping the non-planar target 706. The one or more active elements 704A-N are positioned on the surface of the holder 702 such that their respective transmitting surfaces contact a target surface 710, when the transducer assembly 700 is combined with the non-planar target 706. The holder 702 is combined with the non-planar target 706 such that transmitting surfaces 708 press against the target surface 710. The pressing enables reduction of the space between the transmitting surfaces 708 and the target surface 710, reducing the surface mismatch. It maximizes a surface area of resulting incident surfaces 712 to achieve the surface match ratio close to 1, upon coupling.

In some embodiments, the non-planar target 706 is circular and the holder 702 is a metallic sheet bent in such a way that it forms an incomplete hexagon (with two missing sides) when viewed from the top, such that a distance A is lesser than a diameter D of the non-planar target 706. When the holder 702 and the non-planar target 706 are combined, the difference between the dimensions of the holder 702 and the non-planar target 706 ensures that the transmitting surfaces 708 press against the target surface 710. In some embodiments, the holder 702 is of adjustable or variable length or the holder 702 is combined with the non-planar target 706 with fasteners of adjustable or variable length, to fasten the one or more transducers 714A-N to the non-planar target 706 tightly. The holder 702 may be fabricated with flexible materials like, but not limited to, a fabric, a rubber, a metal or a combination thereof to enable it to envelop the non-planar target 706. The fasteners may include any of, but not limited to, belts, Velcro, hooks, adhesive, and the like, to fasten tightly. The one or more transducers 714A-N are fastened around the non-planar target 706 such that the transmitting surfaces 708 are pressed against the target surface 710.

FIG. 7B illustrates a perspective view of the transducer assembly 700 of FIG. 7A according to some embodiments herein. The transducer assembly 700 may be combined with the non-planar target 706 which is a dialyzer used in a hemodialysis application. The lesser distance A compared to the diameter D of the non-planar target 706 makes the holder 702 function like a fastener which fastens the one or more transducers 714A-N tightly to the non-planar target 706. The holder 702 enables easy attaching and detaching of the transducer assembly 700. The one or more transducers 714A-N may be fastened to a dialyzer for a four-hour dialysis session of one patient. After the dialysis session, the one or more transducers 714A-N may be detached and fastened to another dialyzer for a different patient. The holder 702 may be moved intermittently with respect to the target surface 710 to change the position of the one or more active elements 704. The intermittent change of the position of the transducer assembly 700 exposes different parts of the non-planar target 706 to the ultrasonic waves.

In some embodiments, the target surface 710 functions as a base on which the one or more active elements 704A-N are bonded. The need for a separable holder 702 is eliminated. In case of a wearable artificial kidney, the one or more active elements 704A-N may be bonded directly to the target surface 710 of components of the artificial kidney such that the generated ultrasonic waves are transmitted into the components to improve the efficiency of the purification process.

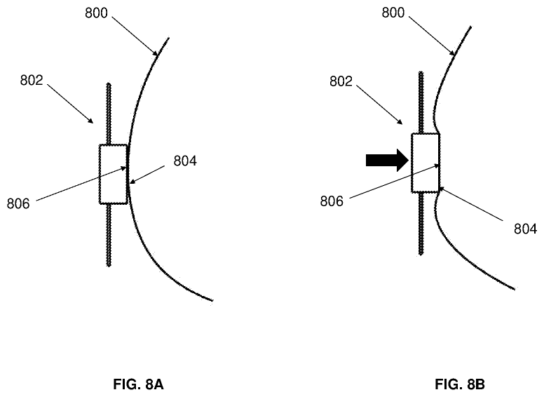

FIGS. 8A and 8B illustrate exemplary side views of a non-planar target surface 800 combined with a transducer assembly 802 according to some embodiments herein. FIG. 8A shows an incident surface 804 that is larger than a transmitting surface 806. Upon pressing the transducer assembly 802 against the target surface 800, the target surface 800 conforms to a profile of the transmitting surface 806 as shown in FIG. 8B. The surface area of the transmitting surface 806 and that of the incident surface 804 become approximately equal, making the surface match ratio very close to 1, that enables efficient transmission of ultrasonic waves into the target surface 800.

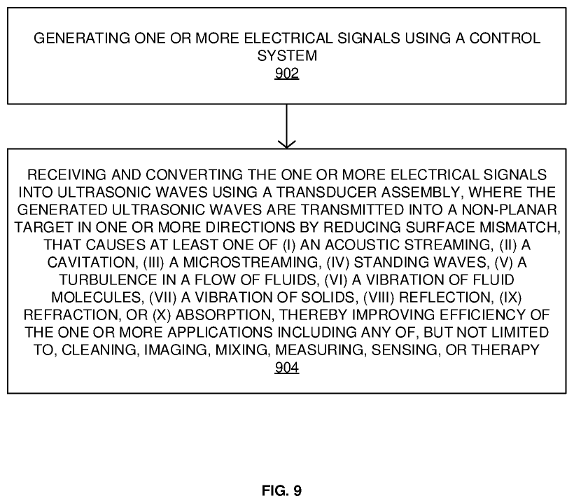

FIG. 9 illustrates a method of generating and transmitting the ultrasonic waves into the non-planar target 302, 402, 508, 606, 706 for the one or more applications according to some embodiments herein. At a step 902, the one or more electrical signals are generated using the control system 308. At a step 904, the one or more electrical signals are received and converted into the ultrasonic waves using the transducer assembly 300, 400, 500, 600, 700, 802. The generated ultrasonic waves are transmitted into the non-planar target 302, 402, 508, 606, 706 in the one or more directions by reducing surface mismatch, that causes at least one of (i) an acoustic streaming, (ii) a cavitation, (iii) a microstreaming, (iv) standing waves, (v) a turbulence in a flow of fluids, (vi) a vibration of fluid molecules, (vii) a vibration of solids, (viii) reflection, (ix) refraction, or (x) absorption, thereby improving efficiency of the one or more applications including any of, but not limited to, cleaning, imaging, mixing, measuring, sensing, or therapy.

1. An apparatus for generating and transmitting ultrasonic waves into a non-planar target for one or more applications, wherein the apparatus comprises:
a control system that generates one or more electrical signals;
a transducer assembly comprising one or more transducers that receive the one or more electrical signals from the control system and convert the one or more electrical signals into the ultrasonic waves,
wherein, by reducing surface mismatch, the generated ultrasonic waves are efficiently transmitted into the non-planar target in one or more directions, wherein the ultrasonic waves cause at least one of (i) an acoustic streaming, (ii) a cavitation, (iii) a microstreaming, (iv) standing waves, (v) a turbulence in a flow of fluids, (vi) a vibration of fluid molecules, (vii) a vibration of solids, (viii) reflection, (ix) refraction, or (x) absorption, thereby improving efficiency of the one or more applications comprising any of, but not limited to, cleaning, imaging, mixing, measuring, sensing, or therapy.

2. The apparatus as claimed in claim 1, wherein the apparatus comprises a holder that envelops the non-planar target and positions each of the one or more transducers around the non-planar target to enable entry of the ultrasonic waves from one or more directions into the non-planar target.

3. The apparatus as claimed in claim 1, wherein a surface geometry of the transducer assembly is matched with a surface geometry of a target surface in order to achieve a surface match ratio close to 1.

4. The apparatus as claimed in claim 1, wherein the one or more transducers comprises one or more active elements, wherein a size of the one or more active elements is limited in order to achieve a surface match ratio close to 1.

5. The apparatus as claimed in claim 1, wherein the one or more active elements are positioned on the holder that enables transmitting surfaces to contact with a target surface when the transducer assembly is combined with the non-planar target.

6. The apparatus as claimed in claim 1, wherein when the transducer assembly is combined with the non-planar target, the transmitting surfaces press against the target surface in order to achieve a surface match ratio close to 1.

7. The apparatus as claimed in claim 2, wherein the holder functions as a base with one or more active elements bonded to the holder.

8. The apparatus as claimed in claim 2, wherein the holder fastens the one or more transducers to the non-planar target.

9. The apparatus as claimed in claim 1, wherein a target surface functions as a base with one or more active elements bonded to the target surface.

10. A method for generating and transmitting ultrasonic waves into a non-planar target for one or more applications, wherein the method comprises:
generating, using a control system, one or more electrical signals;
receiving and converting, using a transducer assembly, the one or more electrical signals into the ultrasonic waves, wherein the generated ultrasonic waves are efficiently transmitted into the non-planar target in one or more directions by reducing surface mismatch, that causes at least one of (i) an acoustic streaming, (ii) a cavitation, (iii) a microstreaming, (iv) standing waves, (v) a turbulence in a flow of fluids, (vi) a vibration of fluid molecules, (vii) a vibration of solids, (viii) reflection, (ix) refraction, or (x) absorption, thereby improving efficiency of the one or more applications comprising any of, but not limited to, cleaning, imaging, mixing, measuring, sensing, or therapy.
